(19) United States
(12) Patent Application Publication
(10) Pub. No.: US 2025/0084365 A1
(43) Pub. Date: Mar. 13, 2025

(54) BACILLUS SUBTILIS FNFH_BS06 AND USE THEREOF

(71) Applicant: ZHEJIANG OCEAN UNIVERSITY, Zhejiang (CN)

(72) Inventors: Yan CHEN, Zhejiang (CN); Tao HAN, Zhejiang (CN); Jiteng WANG, Zhejiang (CN); Shuting HUANG, Zhejiang (CN); Yunping TANG, Zhejiang (CN); Zuisu YANG, Zhejiang (CN); Jialang ZHENG, Zhejiang (CN); Fangmiao YU, Zhejiang (CN)

(73) Assignee: ZHEJIANG OCEAN UNIVERSITY, Zhejiang (CN)

(21) Appl. No.: 18/556,680

(22) PCT Filed: May 24, 2023
(86) PCT No.: PCT/CN2023/095948

§ 371 Date: Oct. 23, 2023

(30) Foreign Application Priority Data
Jun. 2, 2022 (CN) 202210619560.X

(51) Int. Cl. C12N 1/20; A23K 10/12; A23K 10/37; C12R 1/125

(52) U.S. Cl. CPC C12N 1/20; A23K 10/12; A23K 10/37; C12R 2001/125

(57) ABSTRACT

The present invention belongs to the technical field of microorganisms, and provides a Bacillus subtilis FNFH_BS06 and use thereof. On the one hand, the present invention provides a Bacillus subtilis FNFH_BS06, with a preservation number of CGMCC No. 24836, on the other hand, the present invention provides use of the Bacillus subtilis. The strain is inoculated into soybean meal for fermentation, antigen proteins in the soybean meal can be basically completely degraded, the degradation rate of glycinin reaches 95%, the degradation rate of β-conglycinin reaches 90%, and the content of a water-soluble protein increases by 652%, such that the total protein content and protein solubility of fermented soybean meal can be significantly increased, and the protein utilization rate of the soybean meal is significantly improved, thereby improving the nutritional value of the soybean meal, and reaching a leading level in the field of fermentation of soybean meal by microorganisms.

### TECHNICAL FIELD

The present invention belongs to the technical field of microorganisms, and particularly relates to a Bacillus subtilis FNFH_BS06 and use thereof.

### BACKGROUND ART

China has reached a bottleneck in the production of protein feed resources for the long-term dependence on imports. Now, the gap is increasing year by year, the conflicts among people. livestock, aquatic and food have been a serious threat to the national food security strategy. Where, soybean is one of the most important and scarce protein resources that limits the development of China's feed industry. As the largest vegetable protein feed resource in China, soybean meal features high crude protein content, reasonable amino acid composition and low price. However, there are also a variety of anti-nutritional factors in soybean meal, including a large number of protein allergens, non-starch polysaccharides, trypsin inhibitors, lectins and urease, which greatly reduce the nutritive value and protein digestibility, and aggravate the shortage of feedstuff resources. Thus, it is urgent and critical to make the most of the protein feed ingredients in soybean meal, so as to stimulate the development of feed industry.

Among these anti-nutritional factors, antigen protein is characterized by antigenicity, allergenicity, and high thermostability, and accounts for about 30% of the total mass of soybean meal. Without doubt, it is a main factor limiting the efficiency of protein feeding from soybean meal. As the major antigen proteins, glycinin and β-conglycinin show the strongest immunogenicity, and accounts for 70% of the total protein in soybean meal. Glycinin will directly damage and destroy the integrity of intestinal epithelial cells, resulting in hypersensitivity and diarrhea, and going against the growth performance of piglet. B-conglycinin is detrimental to piglet's growth performance as well. Moreover, it will induce allergic reactions in piglets, destroys the immune barrier of duodenal and jejunal mucosa, and promotes the secretion of inflammatory factors. In addition to lowering the nutritional value of feed, such antigen proteins significantly threaten the growth and development of livestock, poultry and aquatic animals. From this point of view, the removal of antigen proteins from soybean meal deserves attention.

Microbial fermentation of raw soybean meal can effectively decompose and destroy the anti-nutritional factors therein. This process has an extensive development and application prospect in feed industry. From pickled tuber mustard, Zhang et al. screened a strain of Bacillus subtilis BS12 that efficiently degraded antigen proteins in soybean meal. After 24 hours of fermentation with this bacterium, 84.8% glycinin and 87.1% β-conglycinin in soybean meal were degraded, while the contents of acid-soluble proteins and crude proteins reached 11.8% and 52.6%, respectively (Zhang et al., 2018). In the similar way, Li et al. screened a strain of Bacillus amyloliquefaciens GYL that efficiently degraded antigen proteins in soybean meal. After 24 hours of fermentation with this bacterium, 92.3% glycinin and 85.1% β-conglycinin in soybean meal were degraded, while the contents of acid-soluble proteins and crude proteins were increased by 997% and 17.5%, respectively, greatly enhancing the nutritional value of soybean meal (Li et al., 2020). Therefore, it is crucial to screen high-quality fermentation strains to effectively remove antigen proteins as well as to enhance the quality of soybean meal through fermentation.

### SUMMARY OF THE INVENTION

The present invention aims to design a technical solution for providing a Bacillus subtilis FNFH_BS06 and use thereof in response to the problems existing in the prior art.

The present invention is specifically implemented by the following technical solution.

A first aspect of the present invention provides a Bacillus subtilis FNFH_BS06. The strain has been preserved in the China General Microbiological Strain Collection Center (CGMCC for short, address: No. 3 Courtyard 1 West Beichen Road, Chaoyang District, Beijing, Institute of Microbiology, Chinese Academy of Sciences) on May 6, 2022, with a preservation number of CGMCC No. 24836.

A second aspect of the present invention provides a bacterial agent containing the Bacillus subtilis FNFH_BS06.

A third aspect of the present invention provides a fermented product obtained by fermentation culture of the Bacillus subtilis FNFH_BS06.

A fourth aspect of the present invention provides use of the Bacillus subtilis FNFH_BS06 or the bacterial agent or the fermented product in efficient degradation of glycinin and/or β-conglycinin.

A fifth aspect of the present invention provides use of the Bacillus subtilis FNFH_BS06 or the bacterial agent or the fermented product in fermentation of soybean meal.

A sixth aspect of the present invention provides use of the Bacillus subtilis FNFH_BS06 or the bacterial agent or the fermented product in degradation of antigen proteins in fermentation of soybean meal.

Further, the antigen proteins comprise glycinin and β-conglycinin.

The Bacillus subtilis strain of the present invention can efficiently hydrolyze glycinin and β-conglycinin by secreting proteases. The strain is inoculated into soybean meal for fermentation for 24 hours at 37° C., antigen proteins in the soybean meal are basically completely degraded, the degradation rate of glycinin reaches 95%, the degradation rate of β-conglycinin reaches 90%, and the content of a water-soluble protein increases by 652%, such that the total protein content and protein solubility of fermented soybean meal can be significantly increased, and the protein utilization rate of the soybean meal is significantly improved, thereby improving the nutritional value of the soybean meal, and reaching a leading level in the field of fermentation of soybean meal by microorganisms.

### BRIEF DESCRIPTION OF THE DRAWINGS

FIG. 1 shows a screening process of Bacillus subtilis FNFH_BS06;

FIG. 2 shows a colony morphology of Bacillus subtilis FNFH_BS06;

FIG. 3 shows a Gram staining microscopic picture of Bacillus subtilis FNFH_BS06; and

FIG. 4 shows time series characterization results of a soybean meal protein degradation degree in a fermentation process of Bacillus subtilis FNFH_BS06.

### DETAILED DESCRIPTION OF THE EMBODIMENTS

In order to fully disclose a Bacillus subtilis strain and use thereof of the present invention, an explanation is given in conjunction with embodiments, but it does not imply any limitation to the present invention.

Embodiment 1: Separation, screening, and identification of Bacillus subtilis FNFH_BS06

Experimental materials:

Mediums:
LB medium: 10 g/L peptone, 5 g/L yeast extract, 5 g/L sodium chloride;
Glycinin screening plate: 0.5 g/L glycinin, 2.5 g/L glucose, 0.2 g/L NaCl, 0.5 g/L K₂HPO₄, 0.2 g/L MgSO₄ 7H₂O, pH 7.0;
β-Conglycinin screening plate: 0.5 g/L β-conglycinin, 2.5 g/L glucose, 0.2 g/L NaCl, 0.5 g/L K₂HPO₄, 0.2 g/L MgSO₄ 7H₂O, pH 7.0.

Experimental method:

1. Separation and screening of Bacillus

5 g of a fermented soybean sample was taken into a 50 mL centrifuge tube with 40 mL of sterile normal saline, subjected to shake culture in a shaker at 37° C. for 30 minutes and then placed in an 80° C. water bath for processing for 20 minutes. Then, the sample solution was diluted with sterile normal saline in a 10-fold gradient. A suitable dilution of bacterial suspension was taken and coated on an LB solid medium plate, with repeating three times for each dilution. Inverted culture was carried out for 24 hours at a constant temperature of 37° C. After colonies grew, strains with Bacillus colony morphological characteristics were selected and subjected to streaking purification.

The separated and purified strains were spot-inoculated onto a glycinin screening plate and a β-conglycinin screening plate, respectively, and subjected to inverted culture for 24 hours at 37° C. Transparent zones around the colonies on the antigen plate mediums were observed, the colony diameter (d) and the transparent zone diameter (D) were measured respectively, and the D/d ratio was calculated. This ratio was used as an evaluation indicator of the ability of the strain to degrade antigen proteins. The single colonies with the highest D/d ratio were selected from the streaked plates for LB liquid culture to prepare a glycerol stock, which was stored in a −80° C. refrigerator.

Methods for preparing glycinin and β-conglycinin were as follows: 5 g of soybean meal was weighed and crushed to be sieved with a 60-mesh sieve, 75 mL of a 0.03 M Tris-HCl buffer with pH 8.5 was added, shake extraction was carried out at 30° C. to 50° C. and 200 rpm for 1 hour, a supernatant was taken after centrifugation was carried out at 9000 rpm for 10 minutes, 0.01 M Na₂SO₃ was added, the pH was regulated to 6.4 with HCl, precipitation was carried out overnight at 4° C., and centrifugation was carried out at 9000 rpm for 10 minutes to obtain a precipitate, which was glycinin. NaCl was added to the separated supernatant to 0.25 M, the pH was regulated to 4 to 6.0 with HCl, shake was carried out for 30 minutes, centrifugation was carried out at 9000 rpm for 10 minutes for separation to obtain a supernatant, the pH was regulated to 4.8, and centrifugation was carried out at 9000 rpm for 10 minutes to obtain a β-conglycinin precipitate. Finally, the glycinin precipitate and the β-conglycinin precipitate were subjected to vacuum freeze drying to obtain a glycinin freeze-dried powder and a β-conglycinin freeze-dried powder.

2. Identification of Bacillus

(1) Gram staining identification

The FNFH_ BS06 single colony was taken and coated onto a glass slide, diluted evenly with normal saline, fixed, and subjected to Gram staining.

(2) Physiological and biochemical identification Referring to the traditional methods of bacterial identification, according to the “Bergey's Manual of Determinative Bacteriology”, the physiological and biochemical characteristics of FNFH_BS06 were detected.

(3) 16S rRNA and gyrB gene sequencing identification

By using modern molecular biology identification techniques, bacterial 16S rRNA sequence identification and Bacillus conserved gene gyrB identification were carried out. By using the 16S rRNA universal primers 27F and 1492R of bacteria as amplification primers and by using the genome of the strain FNFH_BS06 as a template, PCR amplification was performed on a 16S rRNA sequence; at the same time, primers were designed to amplify the Bacillus conserved gene gyrB, and PCR products were recovered and sequenced.

Experimental results:

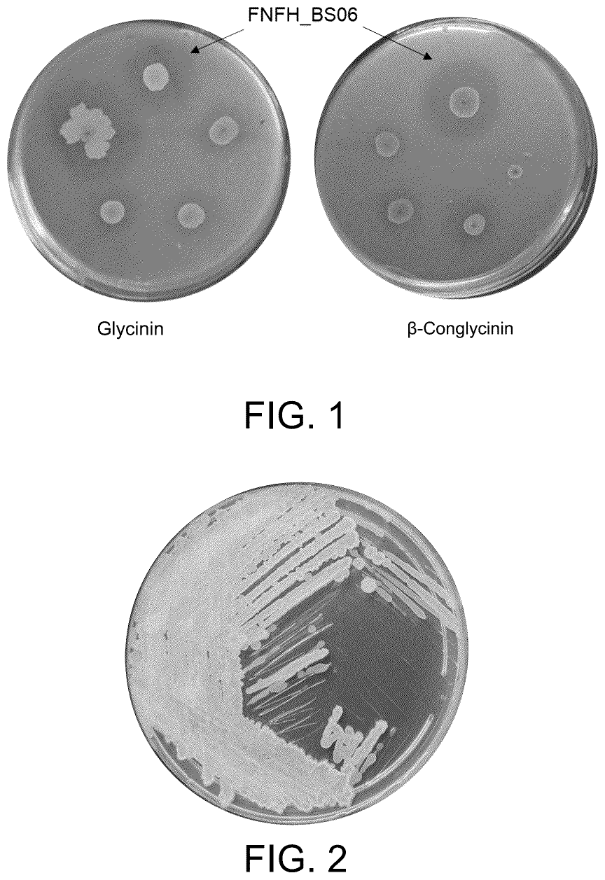

(1) The screening results of the separated strains by using the antigen protein plates are shown in Table 1, where the D/d ratio of Bacillus FNFH_BS06 on the glycinin screening plate and the β-conglycinin screening plate is greater than 2.5, showing stronger antigen protein degradation characteristics. FIG. 1 shows a screening process of Bacillus subtilis FNFH_BS06.

TABLE 1
Screening results of Bacillus on antigen plates

| | Glycinin | | | β-Conglycinin | | |
|---|---|---|---|---|---|---|
| Strain number | D (mm) | d (mm) | D/d | D (mm) | d (mm) | D/d |
| FNFH_BS01 | 42.3 | 29.3 | 1.4 | 14.3 | 12.7 | 1.1 |
| FNFH_BS02 | 22.4 | 13.2 | 1.7 | 6.3 | 5.2 | 1.2 |
| FNFH_BS03 | 21.3 | 11.4 | 1.9 | 15.2 | 8.9 | 1.7 |
| FNFH_BS04 | 17.6 | 9.2 | 1.9 | 11.7 | 10.2 | 1.1 |
| FNFH_BS05 | 10.7 | 8.1 | 1.3 | 11.3 | 10.5 | 1.1 |
| FNFH_BS06 | 30.1 | 10.2 | 3.0 | 37.2 | 14.4 | 2.6 |

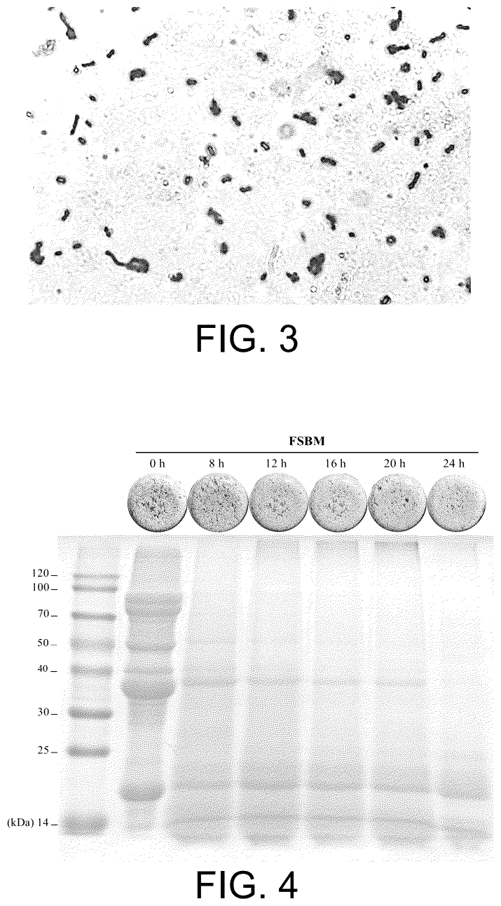

(2) The colony morphology of Bacillus FNFH_BS06 is shown in FIG. 2. The colony is dirty white, opaque and nearly circular, has a moist surface and contains mucus. The Gram staining results are shown in FIG. 3, where FNFH_ BS06 is a Gram-positive bacterium, with a rod-shaped body, arranged in single or paired or chain shapes, and with central spores.

(3) Physiological and biochemical detection

Bacillus FNFH_ BS06 is positive for V-P test and positive for catalase test, can decompose glucose to produce acid, can decompose L-arabinose, cellobiose, D-xylose, sucrose, fructose, maltose, glycerol, D-turanose, mannitol, and sorbitol, and has tolerance to a 7% sodium chloride solution.

(4) The 16S rRNA sequencing results of the Bacillus FNFH_BS06 are shown in the sequence list SEQ ID No. 1. BLAST homology comparison was performed on NCBI. The results show that the sequence homology of 16S rDNA between FNFH_BS06 and strains such as Bacillus subtilis, Bacillus amyloliquefaciens, and Bacillus cereus reaches 100%. The Bacillus conserved gene gyrB sequencing results of FNFH_BS06 are shown in the sequence list SEQ ID No. 2. The BLAST homology comparison results show that the homology between FNFH_BS06 and three strains of Bacillus subtilis species reaches 99.4%. In summary, FNFH_BS06 is identified as Bacillus subtilis.

Meanwhile, the FNFH_BS06 strain has been preserved in the China General Microbiological Strain Collection Center (CGMCC for short, address: No. 3 Courtyard 1 West Beichen Road, Chaoyang District, Beijing, Institute of Microbiology, Chinese Academy of Sciences) on May 6, 2022, with a preservation number of CGMCC No. 24836.

Embodiment 2: Use of Bacillus subtilis FNFH_BS06 in fermentation of soybean meal

### Experimental Materials:

Strain: Bacillus subtilis FNFH_BS06.

### Mediums:

LB medium: 10 g/L peptone, 5 g/L yeast extract, 5 g/L sodium chloride;
GYP medium: 10 g/L glucose, 8 g/L yeast extract, 2 g/L soy peptone, pH 7.0.

### Experimental Method:

1. Pre-treatment of soybean meal: 20 g of soybean meal was weighed and placed in a 250 mL sterilized shake flask whose opening was sealed with a gauze and a sealing film, high-pressure sterilization was carried out at 100° C. for 30 minutes, and cooling was then carried out to a room temperature.
2. Seed preparation: Bacillus subtilis FNFH_BS06 was inoculated onto a 6 mL LB medium and subjected to overnight activation culture at 37° C. and 250 rpm. Then the Bacillus subtilis FNFH_BS06 was transferred to a fresh 30 mL GYP medium with OD=0.5 and cultured at 37° C. and 250 rpm until the logarithmic growth phase.
3. Fermentation inoculation: the seed liquid of Bacillus subtilis FNFH_BS06 cultured until the logarithmic growth phase was mixed with a certain volume of sterile water at an inoculation amount of 3% (v./m.) (the total volume of a mixed seed liquid was 20 mL, even if the initial water content of soybean meal fermentation was 50%), 20 mL of the mixed seed liquid was evenly sprinkled into the pre-treated soybean meal and mixed thoroughly, and then the shake flask was placed in a constant-humidity shaking table for fermentation for 24 hours at 37° C. and 150 rpm.
4. Drying detection: after the fermentation was completed, the soybean meal was taken out and dried at 60° C., then crushed and sieved with a 60-mesh sieve, the contents of glycinin and β-conglycinin, the contents of a crude protein and a water-soluble protein and the degree of soybean meal protein degradation in the product fermented soybean meal obtained by fermentation and the raw material soybean meal were detected, respectively, the degradation rates of glycinin and β-conglycinin were calculated according to the formula, and the protein solubility was calculated according to the formula as follows: Protein solubility=Content of water-soluble protein/Content of crude protein. The results are shown in Table 2.

Content of glycinin: detected by using enzyme linked immunosorbent assay according to the product manual of a glycinin quantitative detection kit purchased from Beijing Longke Fangzhou Biological Engineering Technology Center;

Content of β-conglycinin: detected by using enzyme linked immunosorbent assay according to the product manual of a β-conglycinin quantitative detection kit purchased from Beijing Longke Fangzhou Biological Engineering Technology Center;

Content of crude protein: detected by using the Kjeldahl method (GB/T 6432-2018);

Content of water-soluble protein: 1 g of a sample was weighed and dissolved in 40 mL of double distilled water, subjected to vortex shake for 2 hours, and then centrifuged at 1500 rpm for 10 minutes to collect a supernatant, which was quantified according to the method for determining the content of crude protein, i.e., the Kjeldahl method (GB/T 6432-2018).

Degree of soybean meal protein degradation: 1 g of a sample was weighed and dissolved in 5 mL of an 8 M urea solution, subjected to vortex for 30 minutes, and then centrifuged at 4° C. at 8000 rpm for 5 minutes to separate a supernatant, which was quantified by using a BCA protein quantitative kit, the amount of sample application of the sample was calculated at the 20 μg of protein content, and then the sample was loaded onto polyacrylamide gel for SDS-PAGE analysis.

### Experimental Results:

After the Bacillus subtilis FNFH_BS06 is applied to fermentation of soybean meal, the contents of dry matter, crude protein and water-soluble protein of the obtained product fermented soybean meal are significantly increased, while the content of the antigen proteins are significantly decreased (Table 1). The content of the crude protein was increased by 13.9%, the content of the water-soluble protein was increased by 652.5%, and the corresponding protein solubility was increased by 558.3%, while the content of the glycinin and the content of the β-conglycinin in the antigen proteins are decreased by 95.1% and 90.2%, respectively. This proves that the Bacillus subtilis FNFH_BS06 has a very good degradation effect on the glycinin and β-conglycinin, etc. in the soybean meal, and can significantly increase the content of crude protein, water-soluble protein and the like, thereby improving the protein digestion and utilization rate.

TABLE 2
Analysis of key components of soybean meal before and
after fermentation of Bacillus subtilis FNFH_BS06

| | Raw material | Fermented | Changed |
|---|---|---|---|
| Component | soybean meal | soybean meal | proportion |
| Dry matter, % | 88.16 | 90.12 | |
| Crude protein, % | 46.14 | 52.55 | ↑13.9% |
| Water-soluble | 5.52 | 41.54 | ↑652.5% |
| protein, % | | | |
| Protein solubility, % | 12.0 | 79.0 | ↑558.3% |
| Antigen proteins | | | |
| Glycinin, mg/g | 125.34 | 6.15 | ↑95.1% |
| β-Conglycinin, mg/g | 107.76 | 10.57 | ↑90.2% |

The fermented soybean meal samples of the Bacillus subtilis FNFH_BS06 at 0 h, 8 h, 12 h, 16 h, 18 h, 20 h and 24 h were collected, respectively for SDS-PAGE analysis on the degree of total soybean meal protein degradation. The results are shown in FIG. 4. Compared with the raw material soybean meal, the product fermented soybean meal obtained by fermentation by the Bacillus subtilis FNFH_BS06 gradually degrades within 8 to 24 hours at protein subunit of >25 kDa, while the protein abundance at a molecular weight of <25 kDa gradually increases. At 24 hours of fermentation, the glycinin (30-45 kDa) and the β-conglycinin (50-100 kDa) were basically completely degraded. This proves that the Bacillus subtilis FNFH_BS06 has a very good degradation effect on the antigen proteins in the soybean meal, and can degrade macromolecular insoluble proteins in the soybean meal into micromolecular soluble proteins and peptides within 24 hours, thereby improving the protein bioavailability.

Similarly, the corresponding bacterial agent prepared from the Bacillus subtilis FNFH_BS06 in this embodiment can also achieve the same effect as in this embodiment.

>16S rRNA sequence full length of 1444 bp Bacillus subtilis
CATGGCTCAGGACGAACGCTGGCGGCGTGCCTAATACATGCAAGTCGAGCGGACAG
ATGGGAGCTTGCTCCCTGATGTTAGCGGCGGACGGGTGAGTAACACGTGGGTAACCT
GCCTGTAAGACTGGGATAACTCCGGGAAACCGGGGCTAATACCGGATGGTTGTTTGA
ACCGCATGGTTCAGACATAAAAGGTGGCTTCGGCTACCACTTACAGATGGACCCGCG
GCGCATTAGCTAGTTGGTGAGGTAACGGCTCACCAAGGCGACGATGCGTAGCCGACC
TGAGAGGGTGATCGGCCACACTGGGACTGAGACACGGCCCAGACTCCTACGGGAGG
CAGCAGTAGGGAATCTTCCGCAATGGACGAAAGTCTGACGGAGCAACGCCGCGTGA
GTGATGAAGGTTTTCGGATCGTAAAGCTCTGTTGTTAGGGAAGAACAAGTGCCGTTC
AAATAGGGCGGCACCTTGACGGTACCTAACCAGAAAGCCACGGCTAACTACGTGCCA
GCAGCCGCGGTAATACGTAGGTGGCAAGCGTTGTCCGGAATTATTGGGCGTAAAGGG
CTCGCAGGCGGTTTCTTAAGTCTGATGTGAAAGCCCCCGGCTCAACCGGGGAGGGTC
ATTGGAAACTGGGGAACTTGAGTGCAGAAGAGGAGAGTGGAATTCCACGTGTAGCG
GTGAAATGCGTAGAGATGTGGAGGAACACCAGTGGCGAAGGCGACTCTCTGGTCTG
TAACTGACGCTGAGGAGCGAAAGCGTGGGGAGCGAACAGGATTAGATACCCTGGTA
GTCCACGCCGTAAACGATGAGTGCTAAGTGTTAGGGGGTTTCCGCCCCTTAGTGCTG
CAGCTAACGCATTAAGCACTCCGCCTGGGGAGTACGGTCGCAAGACTGAAACTCAA
AGGAATTGACGGGGGCCCGCACAAGCGGTGGAGCATGTGGTTTAATTCGAAGCAAC
GCGAAGAACCTTACCAGGTCTTGACATCCTCTGACAATCCTAGAGATAGGACGTCCC
CTTCGGGGGCAGAGTGACAGGTGGTGCATGGTTGTCGTCAGCTCGTGTCGTGAGATG
TTGGGTTAAGTCCCGCAACGAGCGCAACCCTTGATCTTAGTTGCCAGCATTCAGTTG
GGCACTCTAAGGTGACTGCCGGTGACAAACCGGAGGAAGGTGGGGATGACGTCAAA
TCATCATGCCCCTTATGACCTGGGCTACACACGTGCTACAATGGACAGAACAAAGGG
CAGCGAAACCGCGAGGTTAAGCCAATCCCACAAATCTGTTCTCAGTTCGGATCGCAG
TCTGCAACTCGACTGCGTGAAGCTGGAATCGCTAGTAATCGCGGATCAGCATGCCGC
GGTGAATACGTTCCCGGGCCTTGTACACACCGCCCGTCACACCACGAGAGTTTGTAA
CACCCGAAGTCGGTGAGGTAACCTTTTA
catggctcaggacgaacgctggcggcgtgcctaatacatgcaagtcgagcggacagatgggagcttgctccctgatgttagcggcggac
gggtgagtaacacgtgggtaacctgcctgtaagactgggataactccgggaaaccggggctaataccggatggttgtttgaaccgcatggt
tcagacataaaaggtggcttcggctaccacttacagatggacccgcggcgcattagctagttggtgaggtaacggctcaccaaggcgacg
atgcgtagccgacctgagagggtgatcggccacactgggactgagacacggcccagactcctacgggaggcagcagtagggaatcttc
cgcaatggacgaaagtctgacggagcaacgccgcgtgagtgatgaaggttttcggatcgtaaagctctgttgttagggaagaacaagtgc
cgttcaaatagggggcaccttgacggtacctaaccagaaagccacggctaactacgtgccagcagccgcggtaatacgtaggtggcaa
gcgttgtccggaattattgggcgtaaagggctcgcaggcggtttcttaagtctgatgtgaaagcccccggctcaaccggggagggtcattg
gaaactggggaacttgagtgcagaagaggagagtggaattccacgtgtagcggtgaaatgcgtagagatgtggaggaacaccagtggc
gaaggcgactctctggtctgtaactgacgctgaggagcgaaagcgtggggagcgaacaggattagataccctggtagtccacgccgtaa
acgatgagtgctaagtgttagggggtttccgccccttagtgctgcagctaacgcattaagcactccgcctggggagtacggtcgcaagact
gaaactcaaaggaattgacgggggcccgcacaagcggtggagcatgtggtttaattcgaagcaacgcgaagaaccttaccaggtcttgac
atcctctgacaatcctagagataggacgtccccttcgggggcagagtgacaggtggtgcatggttgtcgtcagctcgtgtcgtgagatgttg
ggttaagtcccgcaacgagcgcaacccttgatcttagttgccagcattcagttgggcactctaaggtgactgccggtgacaaaccggagga
aggtggggatgacgtcaaatcatcatgccccttatgacctgggctacacacgtgctacaatggacagaacaaagggcagcgaaaccgcg
aggttaagccaatcccacaaatctgttctcagttcggatcgcagtctgcaactcgactgcgtgaagctggaatcgctagtaatcgcggatca
gcatgccgcggtgaatacgttcccgggccttgtacacaccgcccgtcacaccacgagagtttgtaacacccgaagtcggtgaggtaacctt
tta
>gyrB sequence 1004 bp
ACCGCCAAACTTATAAACGCGGAGTTCCGGTTACAGACCTTGAAATCATTGGCGAAA
CGGATCATACAGGAACGACGACACATTTTGTCCCGGACCCTGAAATTTTCTCAGAAA
CAACCGAGTATGATTATGATCTGCTTGCCAACCGCGTACGTGAATTAGCCTTTTTAACA
AAGGGCGTAAACATCACGATTGAGGATAAACGTGAAGGACAAGAGCGCAAAAATGA
ATACCATTACGAAGGCGGAATTAAAAGTTATGTAGAGTATTTAAACCGTTCTAAAGAG
GTTGTCCATGAAGAGCCGATTTACATTGAAGGCGAAAAGGACGGCATAACGGTTGAA
GTAGCTTTGCAATACAATGACAGCTACACAAGCAACATTTACTCGTTTACAAACAACA
TTAACACGTACGAAGGCGGTACCCATGAAGCTGGCTTTAAAACGGGCCTGACTCGTG
TTATCAACGATTACGCCAGAAAAAAAGGGCTTATTAAAGAAAATGATCCAAACCTAA
GCGGAGATGACGTAAGGGAAGGGCTGACAGCGATTATTTCAATCAAACACCCTGATC
CGCAGTTTGAGGGCCAAACGAAAACAAAGCTGGGCAACTCAGAAGCACGGACGAT
CACCGATACGTTATTTTCTGCGGCGATGGAAACATTTATGCTGGAAAATCCAGATGCG
GCCAAAAAAATTGTCGATAAAGGCTTAATGGCGGCAAGAGCAAGAATGGCTGCGAA
AAAAGCGCGTGAACTAACACGCCGTAAGAGTGCTTTGGAAATTTCAAACCTGCCCG
GTAAGTTAGCGGACTGCTCTTCAAAAGATCCGAGCATCTCCGAGTTATATATCGTAGA
GGGTGACTCTGCCGGAGGATCTGCTAAACAAGGACGCGACAGACATTTCCAAGCCAT
TTTGCCGCTTAGAGGTAAGATCCTAAACGTTGAAAAGGCCAGACTGGATAAAATCCT
TTCTAACAACGAAGTTCGCTCTATGATCACAGCG
accgccaaacttataaacgcggagttccggttacagaccttgaaatcattggcgaaacggatcatacaggaacgacgacacattttgtcccg
gaccctgaaattttctcagaaacaaccgagtatgattatgatctgcttgccaaccgcgtacgtgaattagcctttttaacaaagggcgtaaaca
tcacgattgaggataaacgtgaaggacaagagcgcaaaaatgaataccattacgaaggcggaattaaaagttatgtagagtatttaaaccgtt
ctaaagaggttgtccatgaagagccgatttacattgaaggcgaaaaggacggcataacggttgaagtagctttgcaatacaatgacagctac
acaagcaacatttactcgtttacaaacaacattaacacgtacgaaggggtacccatgaagctggctttaaaacgggcctgactcgtgttatc
aacgattacgccagaaaaaaagggcttattaaagaaaatgatccaaacctaagcggagatgacgtaagggaagggctgacagcgattattt
caatcaaacaccctgatccgcagtttgagggccaaacgaaaacaaagctgggcaactcagaagcacggacgatcaccgatacgttattttc
tgcggcgatggaaacatttatgctggaaaatccagatgcggccaaaaaaattgtcgataaaggcttaatggcggcaagagcaagaatggct
gcgaaaaaagcgcgtgaactaacacgccgtaagagtgctttggaaatttcaaacctgcccggtaagttagcggactgctcttcaaaagatc
cgagcatctccgagttatatatcgtagagggtgactctgccggaggatctgctaaacaaggacgcgacagacatttccaagccattttgccg
cttagaggtaagatcctaaacgttgaaaaggccagactggataaaatcctttctaacaacgaagttcgctctatgatcacagcg

1. A Bacillus subtilis FNFH_BS06, with a preservation number of CGMCC No. 24836.

2. A bacterial agent containing the Bacillus subtilis FNFH_BS06 of claim 1.

3. A fermented product obtained by fermentation culture of the Bacillus subtilis FNFH_BS06 of claim 1.

4. Use of the Bacillus subtilis FNFH_BS06 of claim 1 in degradation of glycinin and/or β-conglycinin.

5. Use of the Bacillus subtilis FNFH_BS06 of claim 1 in fermentation of soybean meal.

6. Use of the Bacillus subtilis FNFH_BS06 of claim 1 in degradation of antigen proteins in fermentation of soybean meal.

7. The use of claim 6, wherein the antigen proteins comprise glycinin and β-conglycinin.

8. Use of the bacterial agent of claim 2 in degradation of glycinin and/or β-conglycinin.

9. Use of the fermented product of claim 3 in degradation of glycinin and/or β-conglycinin.

10. Use of the bacterial agent of claim 2 in fermentation of soybean meal.

11. Use of the fermented product of claim 3 in fermentation of soybean meal.

12. Use of the bacterial agent of claim 2 in degradation of antigen proteins in fermentation of soybean meal.

13. The use of claim 12, wherein the antigen proteins comprise glycinin and β-conglycinin.

14. Use of the fermented product of claim 3 in degradation of antigen proteins in fermentation of soybean meal.

15. The use of claim 14, wherein the antigen proteins comprise glycinin and β-conglycinin.


SEQUENCE LISTING (2 sequences)

SEQ ID NO: 1 - RNA, 1444 nt, Bacillus subtilis
  source 1..1444: mol_type = genomic RNA
catggctcaggacgaacgctggcggcgtgcctaatacatgcaagtcgagcggacagatgggagcttgctccctgatgttagcggcggacgggtgagtaacacgtgggtaacctgcctgtaagactgggataactccgggaaaccggggctaataccggatggttgtttgaaccgcatggttcagacataaaaggtggcttcggctaccacttacagatggacccgcggcgcattagctagttggtgaggtaacggctcaccaaggcgacgatgcgtagccgacctgagagggtgatcggccacactgggactgagacacggcccagactcctacgggaggcagcagtagggaatcttccgcaatggacgaaagtctgacggagcaacgccgcgtgagtgatgaaggttttcggatcgtaaagctctgttgttagggaagaacaagtgccgttcaaatagggcggcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactggggaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaatcctagagataggacgtccccttcgggggcagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggcagcgaaaccgcgaggttaagccaatcccacaaatctgttctcagttcggatcgcagtctgcaactcgactgcgtgaagctggaatcgctagtaatcgcggatcagcatgccgcggtgaatacgttcccgggccttgtacacaccgcccgtcacaccacgagagtttgtaacacccgaagtcggtgaggtaacctttta

SEQ ID NO: 2 - DNA, 1004 bp, Bacillus subtilis
  source 1..1004: mol_type = genomic DNA
accgccaaacttataaacgcggagttccggttacagaccttgaaatcattggcgaaacggatcatacaggaacgacgacacattttgtcccggaccctgaaattttctcagaaacaaccgagtatgattatgatctgcttgccaaccgcgtacgtgaattagcctttttaacaaagggcgtaaacatcacgattgaggataaacgtgaaggacaagagcgcaaaaatgaataccattacgaaggcggaattaaaagttatgtagagtatttaaaccgttctaaagaggttgtccatgaagagccgatttacattgaaggcgaaaaggacggcataacggttgaagtagctttgcaatacaatgacagctacacaagcaacatttactcgtttacaaacaacattaacacgtacgaaggcggtacccatgaagctggctttaaaacgggcctgactcgtgttatcaacgattacgccagaaaaaaagggcttattaaagaaaatgatccaaacctaagcggagatgacgtaagggaagggctgacagcgattatttcaatcaaacaccctgatccgcagtttgagggccaaacgaaaacaaagctgggcaactcagaagcacggacgatcaccgatacgttattttctgcggcgatggaaacatttatgctggaaaatccagatgcggccaaaaaaattgtcgataaaggcttaatggcggcaagagcaagaatggctgcgaaaaaagcgcgtgaactaacacgccgtaagagtgctttggaaatttcaaacctgcccggtaagttagcggactgctcttcaaaagatccgagcatctccgagttatatatcgtagagggtgactctgccggaggatctgctaaacaaggacgcgacagacatttccaagccattttgccgcttagaggtaagatcctaaacgttgaaaaggccagactggataaaatcctttctaacaacgaagttcgctctatgatcacagcg